(19) United States
(12) Patent Application Publication
(10) Pub. No.: US 2010/0008860 A1
(43) Pub. Date: Jan. 14, 2010

(54) COLOR-MARKED OLIGOSACCHARIDES OR POLYSACCHARIDES

(75) Inventors: Jorg Kowalczyk, Eisenberg/Steinborn (DE); Thomas Rose, Worms (DE); Stephan Hausmanns, Wiesbaden (DE); Thomas Walter, Muhlhausen (DE); Wolfgang Wach, Worms (DE); Norbert Gretz, Mannheim (DE); Maliha Sadick, Hassloch (DE); Daniel Schock, Mannheim (DE)

Correspondence Address: HARNESS, DICKEY & PIERCE, P.L.C., P.O. BOX 828, BLOOMFIELD HILLS, MI, 48303, US

(73) Assignee: UNIVERSITAT HEIDELBERG, Heidelberg (DE)

(21) Appl. No.: 12/525,992

(22) PCT Filed: Jan. 23, 2008
(86) PCT No.: PCT/EP2008/000479

§ 371 Date: Aug. 5, 2009

(30) Foreign Application Priority Data
Feb. 9, 2007 (DE) 10 2007 007 738.8

(51) Int. Cl. A61K 49/00; C08B 37/00

(52) U.S. Cl. 424/9.6; 536/123.1; 536/54

(57) ABSTRACT

The invention concerns color-marked oligosaccharides or polysaccharides, procedures for the synthesis of these color-marked oligosaccharides or polysaccharides and their use in the determination of kidney function.

The invention concerns color-marked oligosaccharide or polysaccharides, procedures for synthesizing these color-marked oligosaccharides or polysaccharides, their use for the synthesis of an agent for the determination of kidney function and its use for assessing kidney function.

In kidney diagnostics, the quantitative and qualitative functional review plays a large role. Among other things, the glomerular filtration rate is measured. The glomerular filtration rate is an important element for assessing kidney function. It can be determined at least approximately from the renal clearance. The renal clearance describes the distance of a certain exogenous or endogenous substance of the blood as specific performance of the kidney. For determination of the renal clearance and thus the glomerular filtration rate, various indicator substances are known. Thereby, the indicator substance used most frequently is creatinine. An additional example for an endogenous indicator substance is cystatin C. An exogenous indicator substance is inulin. Gretz et al (Pediatr. Nephrol. 22 (2207); 167-169) describes various methods of measurement of the glomerular filtration rate.

U.S. Pat. No. 6,995,019 B2 describes an indicator substance in which fluorescein isothiocyanate (FITC) is bound to sinestrin. Even inulin coupled to FITC is known from Lorenz and Gruenstein (Renal Physiol. 45 (1999); 172-177).

The emission maximum of FITC lies at 520 nm. Hemoglobin shows very strong absorption at 520 nm. In the measurement of glomerular filtration rates with FITC-marked indicator substances in the presence of hemoglobin, for example, in hemolytic processes, this leads to errors and imprecision.

The technical problem on which the invention is based is therefore the provision of indicator substances that make it possible to perform precise measurements of the renal clearance, whereby the presence of hemoglobin does not have a disruptive effect on the measurement.

A different problem on which the invention is based is the provision of improved indicator substances for the measurement of renal clearance.

The invention solves the technical problem on which it is based by color-marked oligosaccharides or polysaccharides according to the independent claims, particularly by color-marked oligosaccharides or polysaccharides, comprising an oligosaccharide or polysaccharide coupled with a pigment, whereby the pigment has an emission maximum of at least 725 nm and at most 875 nm. Further, the present invention solves the technical problem on which it is based by color-marked oligosaccharides or polysaccharides that comprise an oligosaccharide or polysaccharide that is coupled with a cyanine pigment.

The invention also solves the problem on which it is based by providing a color-marked oligosaccharide or polysaccharide, comprising an oligosaccharide or polysaccharide coupled with a cyanine pigment, whereby the cyanine pigment has an emission maximum of at least 725 nm and at most 875 nm.

For the determination of the glomerular filtration rate, indicator substances must be used that are excreted by the kidney. The kidney excretes hydrophilic substances. Surprisingly, it was shown that hydrophobic pigments are either generally excreted by the kidney or are particularly well excreted when they are bound to certain hydrophilic oligosaccharides or polysaccharides. Particularly, it was shown that in a preferred embodiment, the usability of color-marked indicator substances can be influenced by the length of the chain of the oligosaccharide or polysaccharide.

Further, it was surprisingly shown that in a preferred embodiment, the half-life of the pigment can be adjusted by the selection of certain oligosaccharides or polysaccharides. Thus, for example, the half-life of color-marked 1-F-fructofuranosyl-d-nystose is higher than the half-life of color-marked sinistrin.

Beyond that, it was shown in a preferred embodiment that even oligosaccharides', particularly with a DP of 2 to 10 and even with a DP of 2 to 7 with color-marking, can be used as an indicator substance.

The color-marked oligosaccharides and polysaccharides in accordance with the invention make precise and time-saving procedures for measuring kidney functions possible.

In accordance with the invention, preferably the color-marked oligosaccharide or polysaccharide is an oligosaccharide. In accordance with the invention, preferably the color-marked oligosaccharide or polysaccharide is a polysaccharide.

Preferably, in accordance with the invention, the oligosaccharide or polysaccharide has a degree of polymerization, also called DP, of 2 to 65. In accordance with the invention, preferably, the oligosaccharide or polysaccharide has a DP of 3 to 65. Preferably, in accordance with the invention the oligosaccharide or polysaccharide has a DP of 4 to 65. Preferably, according to the invention, the oligosaccharide or polysaccharide has a DP of 5 to 65. Preferably, in accordance with the invention, the oligosaccharide or polysaccharide has a DP of 6 to 65.

Preferred in accordance with the invention, the oligosaccharide or polysaccharide has a DP of 2 to 50. Preferred in accordance with the invention, the oligosaccharide or polysaccharide has a DP of 3 to 50. Preferred in accordance with the invention, the oligosaccharide or polysaccharide has a DP of 4 to 50. Preferred in accordance with the invention, the oligosaccharide or polysaccharide has a DP from 5 to 50.

Preferred in accordance with the invention, the oligosaccharide or polysaccharide has a DP of 2 to 40. Preferred in accordance with the invention, the oligosaccharide or polysaccharide has a DP of 3 to 40. Preferred in accordance with the invention, the oligosaccharide or polysaccharide has a DP of 4 to 40. Preferred in accordance with the invention, the oligosaccharide or polysaccharide has a DP from 5 to 40. Preferred in accordance with the invention, the oligosaccharide or polysaccharide has a DP from 10 to 40.

Preferred in accordance with the invention, the oligosaccharide or polysaccharide has a DP of 2 to 25. Preferred in accordance with the invention, the oligosaccharide or polysaccharide has a DP of 3 to 25. Preferred in accordance with the invention, the oligosaccharide or polysaccharide has a DP of 4 to 25. Preferred in accordance with the invention, the oligosaccharide or polysaccharide has a DP from 5 to 25.

Preferred in accordance with the invention, the oligosaccharide or polysaccharide has a DP of 2 to 10. Preferred in accordance with the invention, the oligosaccharide or polysaccharide has a DP of 3 to 10. Preferred in accordance with the invention, the oligosaccharide or polysaccharide has a DP of 4 to 10. Preferred in accordance with the invention, the oligosaccharide or polysaccharide has a DP from 5 to 10. Preferred in accordance with the invention, the oligosaccharide or polysaccharide has a DP from 6 to 10.

Preferred in accordance with the invention, the oligosaccharide or polysaccharide has a DP of at least 2. Preferred in accordance with the invention, the oligosaccharide or polysaccharide has a DP of at least 3. Preferred in accordance with the invention, the oligosaccharide or polysaccharide has a DP of at least 4. Preferred in accordance with the invention, the oligosaccharide or polysaccharide has a DP of at least 5. Preferred in accordance with the invention, the oligosaccharide or polysaccharide has a DP of at least 6. Preferred in accordance with the invention, the oligosaccharide or polysaccharide has a DP of at least 7. Preferred in accordance with the invention, the oligosaccharide or polysaccharide has a DP of at least 8. Preferred in accordance with the invention, the oligosaccharide or polysaccharide has a DP of at most 10. Preferred in accordance with the invention, the oligosaccharide or polysaccharide has a DP of at most 25. Preferred in accordance with the invention, the oligosaccharide or polysaccharide has a DP of at most 30. Preferred in accordance with the invention, the oligosaccharide or polysaccharide has a DP of at most 40. Preferred in accordance with the invention, the oligosaccharide or polysaccharide has a DP of at most 50. Preferred in accordance with the invention, the oligosaccharide or polysaccharide has a DP of at most 65.

Preferred, in accordance with the invention, the oligosaccharide or polysaccharide has a DP of 2 or 3. Preferred in accordance with the invention the oligosaccharide or polysaccharide has a DP of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65.

Preferred, in accordance with the invention, the oligosaccharide or polysaccharide has a DP of over 65.

In the context of the present invention, an oligosaccharide is understood to be a hydrocarbon that consists of approximately 10, especially of 2 to 10, very especially out of 2 to 7 monosaccharide units. In the context of the present invention, a polysaccharide is understood to be a hydrocarbon that consists of at least 10, preferably more than 10 monosaccharide units.

In accordance with the invention, the oligosaccharide preferably has a DP of 2.

In accordance with the invention, the oligosaccharide preferably has a DP of 3.

In accordance with the invention, the oligosaccharide preferably has a DP of 4.

In accordance with the invention, the oligosaccharide preferably has a DP of 5.

In accordance with the invention, the oligosaccharide preferably has a DP of 6.

In accordance with the invention, the oligosaccharide preferably has a DP of 7.

In accordance with the invention, the oligosaccharide preferably has a DP of 8.

In accordance with the invention, the oligosaccharide preferably has a DP of 9.

In accordance with the invention, the oligosaccharide preferably has a DP of 10.

In accordance with the invention, the oligosaccharide preferably has a DP from 2 to 10. In accordance with the invention, the oligosaccharide preferably has a DP from 3 to 10. In accordance with the invention, the oligosaccharide preferably has a DP from 4 to 10. In accordance with the invention, the oligosaccharide preferably has a DP from 5 to 10. In accordance with the invention, the oligosaccharide preferably has a DP from 2 to 7. In accordance with the invention, the oligosaccharide preferably has a DP from 3 to 7. In accordance with the invention, the oligosaccharide preferably has a DP from 4 to 7. In accordance with the invention, the oligosaccharide preferably has a DP from 5 to 7.

In accordance with the invention, the polysaccharide preferably has a DP of at least 10. In accordance with the invention, the polysaccharide preferably has a DP of at least 11. In accordance with the invention, the polysaccharide preferably has a DP from 10 to 65. In accordance with the invention, the polysaccharide preferably has a DP from 11 to 65. In accordance with the invention, the polysaccharide preferably has a DP from 20 to 65. In accordance with the invention, the polysaccharide preferably has a DP from 40 to 65. In accordance with the invention, the polysaccharide preferably has a DP of 10. In accordance with the invention, the polysaccharide preferably has a DP 11. In accordance with the invention, the polysaccharide preferably has a DP of 12. In accordance with the invention, the polysaccharide preferably has a DP between 10 and 12.

In accordance with the invention the oligosaccharide or polysaccharide is preferably branched. In accordance with the invention, the oligosaccharide is preferably branched. In accordance with the invention, the polysaccharide is preferably branched.

In accordance with the invention, the oligosaccharide or polysaccharide is preferably not branched. In accordance with the invention, the oligosaccharide is preferably not branched. In accordance with the invention, the polysaccharide is preferably not branched.

In accordance with the invention, the oligosaccharide or the polysaccharide is preferably selected from a group consisting of oligofructanes, polyfructanes, inulin, sinistrin and insoluble maltodextrines or dextrines, that have in addition to the α-1,4 bond, at least one α-1,3, α-1,2- or/and an α-1,6 bond.

In accordance with the invention, preferably the fructose molecules in the oligofructanes or in the polyfructanes are bound to each other by β-2-1 bonds. Preferred, in accordance with the invention the fructose molecules in the oligofructanes are bound to each other by β-2,1 bonds. Preferred, in accordance with the invention, the fructose molecules in the polyfructanes are bound to each other by β-2,1 bonds.

In accordance with the invention, preferably, the oligofructanes are oligofructose and/or fructooligosaccharides (FOS. Preferred, according to the invention, the fructooligosaccharides are of the type GFₙ (glucose-fructoseₙ) or of the type Fₙ (Fructoseₙ). Preferred in accordance with the invention, the fructooligosaccharides contain a terminal glucose molecule. It can also be provided that the fructoseoligosaccharides contain a terminal fructose molecule in pyranose structure. Preferably, according to the invention, the fructooligosaccharides do not contain a terminal glucose molecule.

A source for obtaining the oligosaccharides and polysaccharides used in accordance with the invention can, for example, be fructane-inulin (“alan” starch) CAS number 9005-80-5), that is stored by many plants, for example blue sailors/chicory as a reserve substance/reserve carbohydrate. From chicory, for example, chicorium intybus, inulin is obtained by watery extraction and additional purification steps. A synthesis that is independent of plants or of the enzymatic structure of fructanes is, for example, also possible with the help of bacterial fructosyl transferases.

To the extent product mixtures are created thereby, the desired molecules can be separated from such using conventional procedures. Inulin, which can be made with the help of bacterial fructosyl transferases has, as a rule, a long chain, but more branches (β(2→6) branches within the chain of the fructose molecules) as vegetable inulin. Preferred, in accordance with the invention are polyfructanes of the type GFₙ or of the type Fₙ. Preferred, in accordance with the invention, the polyfructanes contain a terminal glucose molecule. Preferred, in accordance with the invention, the polyfructanes do not contain a terminal glucose molecule.

Preferred, in accordance with the invention is the oligosaccharide or the polysaccharide sinistrin. Preferred, according to the invention is the oligosaccharide or the polysaccharide inulin. Preferred, according to the invention, the oligosaccharide or the polysaccharide is an oligofructane. Preferred, in accordance with the invention, the oligosaccharide or the polysaccharide is a polyfructane. Preferred, according to the invention, the oligosaccharide is an oligofructane. Preferred, according to the invention, the polysaccharide, is a polyfructane.

Preferred, according to the invention, the oligosaccharide or the polysaccharide, is an insoluble maltodextrin or dextrin that has, in addition to the α-1,4 bond, at least one α-1,3, α-1,2 or/and an α-1,6 bond. Preferred, according to the invention, the oligosaccharide is a maltodextrin or dextrin that has, in addition to the α-1,4 bond at least one α-1,3, α-1,2 or/and an α-1,6 bond. Preferred, according to the invention, the polysaccharide is a maltodextrin or dextrin that has, in addition to the α-1,4 bond at least one α-1,3, α-1,2 or/and an α-1,6 bond.

Preferred, according to the invention, the oligosaccharide or the polysaccharide, is an insoluble maltodextrin that has, in addition to the α-1,4 bond at least one α-1,3, α-1,2 or/and an α-1,6 bond. In accordance with the invention, the oligosaccharide is preferably a maltodextrin that has, in addition to the α-1,4 bond at least one α-1,3, α-1,2 or/and an α-1,6 bond. In accordance with the invention, the polysaccharide is preferably a maltodextrin that has, in addition to the α-1,4 bond at least one α-1,3, α-1,2 or/and an α-1,6 bond. In accordance with the invention, the oligosaccharide or the polysaccharide is preferably an insoluble dextrin that has, in addition to the α-1,4 bond at least one α-1,3, α-1,2 or/and an α-1,6 bond. In accordance with the invention, the oligosaccharide is preferably a dextrin that has, in addition to the α-1,4 bond at least one α-1,3-α-1,2 or/and an α-1,6 bond. In accordance with the invention, the polysaccharide is preferably a dextrin that has, in addition to the α-1,4 bond at least one α-1,3, α-1,2 or/and an α-1,6 bond.

In accordance with the invention, the oligosaccharide is preferably fructofuranosyl-d-nystose.

In accordance with the invention, the oligosaccharide is preferably kestose. In accordance with the invention, the oligosaccharide is preferably nystose.

In accordance with the invention, the oligosaccharide is preferably of Type F₄.

In accordance with the invention, the oligosaccharide is preferably of Type F₅.

In accordance with the invention, the oligosaccharide is preferably of Type GF₃.

In accordance with the invention, the oligosaccharide is preferably of Type GF₄.

In accordance with the invention, the oligosaccharide preferably consists of 4 fructose molecules. In accordance with the invention, the oligosaccharide preferably consists of 5 fructose molecules. In accordance with the invention, the oligosaccharide preferably consists of 3 fructose molecules and one glucose molecule. In accordance with the invention, the oligosaccharide preferably consists of 4 fructose molecules and one glucose molecule. In accordance with the invention the oligosaccharide preferably has a DP of 4. In accordance with the invention, the oligosaccharide preferably has a DP of 5.

In accordance with the invention, the oligosaccharide or polysaccharide is a polyfructosane. In accordance with the invention, the oligosaccharide or polysaccharide preferably consists of fructose units. In accordance with the invention, the oligosaccharide or polysaccharide preferably contains mostly fructose units. In accordance with the invention, the oligosaccharide or polysaccharide preferably contains essentially no glucose units, particularly no glucose units. In accordance with the invention, the oligosaccharide or polysaccharide preferably contains only terminal glucose units. In accordance with the invention, the oligosaccharide or polysaccharide preferably contains glucose at an overall proportion of at most 0 to 10%, particularly preferred of at most 1 to 10%, even more preferred of at most 1 to 5%, particularly at most 2 to 3%, especially of at most 1%. In accordance with the invention the oligosaccharide or polysaccharide preferably contains glucose at an overall proportion of at most 0 to 5%. In accordance with the invention, the oligosaccharide or polysaccharide preferably contains a total share of glucose of at most 0 to 3%.

Synthesis of the oligosaccharides and polysaccharides for color-marking preferably takes place by chromatographic separation of the desired substances from mixtures that already contain them.

In accordance with the invention, the pigment that is coupled to the oligosaccharide or polysaccharide preferably has an emission maximum of at least 725 nm and at most 875 nm. In accordance with the invention, the pigment preferably has an emission maximum of at least 750 nm and at most 850 nm. In accordance with the invention, the pigment preferably has a maximum emission of at least 775 and at most 825 nm. In accordance with the invention the pigment preferably has a maximum emission of at least 790 and at most 810 nm. In accordance with the invention, preferably, the pigment has maximum emission of at least 700 nm. In accordance with the invention, preferably, the pigment has an emission maximum of at least 725 nm. In accordance with the invention, the pigment preferably has an emission maximum of at least 750 nm.

In accordance with the invention, the pigment preferably has an emission maximum of approximately 775 nm. In accordance with the invention, the pigment preferably has an emission maximum of approximately 800 nm. In accordance with the invention, the pigment preferably has an emission maximum of 800 nm.

The determination of the emission maximum of the pigment that is coupled to the oligosaccharide or polysaccharide, particularly a florescent pigment preferably takes place in accordance with the invention as per the method that is described in the European pharmacopeia. The determination of the emission maximum of the pigment coupled to the oligosaccharide or polysaccharide, particularly a florescent pigment preferably takes place in the style of the method described in the European pharmacopeia. In accordance with the invention, the solution of the pigment is preferably filled into a suitable vessel, irradiated with monochromatic light and the intensity of the fluorescent rays emanating from the probe are subsequently measured at an angle of 90° to the incoming stream of light. As a device for this measurement, a Hitachi Fluorometer F-3010 can be used, for example. In accordance with the invention, preferably the measurement is performed at room temperature, particularly at 25° C. In accordance with the invention, the concentration of the probe solution is preferably between 5 and 50 μg/ml, particularly at approximately 30 μg/ml or 10⁻⁶ mol/l.

In the context of this invention, by pigment, a pigment is understood, especially a pigment that was mentioned above with the identified emission maxima.

In accordance with the invention, preferably the pigment is a cyanine pigment.

In accordance with the invention, preferably the pigment is a fluorescent pigment.

In accordance with the invention, preferably, the pigment is not fluorescein isothiocyanate, also known to the expert as FITC. In accordance with the invention, the pigment is preferably not a FITC derivative.

In accordance with the invention, the pigment is a mono-functional or bi-functional isothiocyanate cyanine pigment. In accordance with the invention, preferably the pigment is an isothiocyanate cyanine pigment. In accordance with the invention, the pigment is preferably a mono-functional isothiocyanate cyanine pigment. In accordance with the invention, the pigment is preferably a bi-functional isothiocyanate cyanine pigment. In accordance with the invention, the pigment is preferably an isothiocyanate cyanine derivative.

In accordance with the invention, the pigment is an indocyanine green derivative. In accordance with the invention, the pigment is preferably indocyanine green.

In accordance with the invention, a color-marked oligosaccharide or polysaccharide with the structure CY-ITC-SAC, whereby CY is a cyanine pigment, ITC is an isothiocyanate and SAC is an oligosaccharide or a polysaccharide. In accordance with the invention, preferably CY is a pigment that has been revealed here. In accordance with the invention, CY is preferably an indocyanine green derivative. In accordance with the invention, preferably CY is indocyanine green.

In accordance with the invention, a color-marked oligosaccharide or polysaccharide is preferred, whereby the pigment is indocyanine green, and the indocyanine green derivative is synthesized from indocyanine green and isothiocyanate.

In accordance with the invention, the pigment is preferably a CY pigment. CY pigments and their nomenclature are described in Mujumdar et al., Cytometry 10, (1989); page 11-19 and in Ernst et al., Cytometry 10 (1989); page 3-10.

In accordance with the invention, the pigment is preferably CY7.4. In accordance with the invention, the pigment is preferably CY7.4-iodoacetamide. In accordance with the invention, preferably the pigment is CY7.4-isothionate.

In accordance with the invention, preferably the pigment has a structure as per Formula I:

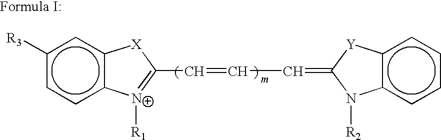

X, Y, R₁, R₂ and R₃, in accordance with the invention, can preferably be various groups in Formula 1. It is preferred, in accordance with the invention that m is 1, 2 or 3. Preferably, in accordance with the invention, m is 3. According to the invention it is preferred that X is S, O, an N—R— or an isopropylide group. According to the invention it is preferred that Y is S, O, an N—R or an isopropylide group. In accordance with the invention, preferably R₁ is a methyl group. In accordance with the invention, preferably R₂ is a sulfopropyl group or a sulfobutyl group. In accordance with the invention, preferably R₂ is a sulfopropyl group. In accordance with the invention, preferably R₂ is a sulfobutyl group. In accordance with the invention, preferably R₃ is ITC, H, NH₂, NCS, NHBOC or NHCSNHCH₃. In accordance with the invention, preferably R₃=ITC. In accordance with the invention, preferably R₃=NHBOC, i.e. a 5-t-butyl carbamido group. In accordance with the invention, preferably R₃═NH₂. In accordance with the invention, preferably R₃═NCS. In accordance with the invention, preferably R₃═NHCSNHCH₃.

In accordance with the invention, preferably, the pigment has a structure according to Formula II:

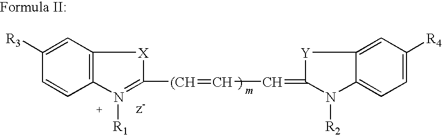

X, Y, R₁, R₂, R₃ and R₄ in formula II can preferably be various groups according to the invention. According to the invention, preferably m is 1, 2 or 3. According to the invention, preferably m is 3. According to the invention, preferred X is S, O, an N—R or an isopropylide group. According to the invention, preferred is Y is S, O, an N—R or an isopropylide group. According to the invention R₁ is preferably a methyl group. According to the invention, R₂ is preferably a sulfopropyl group or a sulfobutyl group. According to the invention R₂ is preferably a sulfopropyl group. According to the invention, R₂ is preferably a sulfobutyl group. According to the invention, R₃ is ITC, H, NH₂, NCS, NHBOC or NHCSNHCH₃. In accordance with the invention, preferably R₃=ITC. In accordance with the invention, preferably R₃═NHBOC, i.e. a 5-t-butyl carbamido group. In accordance with the invention, preferably R₃═NH₂. In accordance with the invention, preferably R₃═NCS. According to the invention, preferably, R₃═NHCSNHCH₃. According to the invention, preferably R₄ is ITC, H, NH₂, NCS, NHBOC or NHCSNHCH₃. According to the invention, preferably R₄=ITC. According to the invention, preferably R₄═NHBOC, i.e. a 5-t-butyl carbamido group. According to the invention, R₄═NH₂.

Preferred in accordance with the invention is R4=NCS. Preferred according to the invention is R4=NHCSNHCH₃.

According to the invention, preferably the pigment is CY7.8. According to the invention, preferably the pigment is CY7.8-isothiocyanate.

According to the invention, preferably the pigment has a structure as per Formula III.

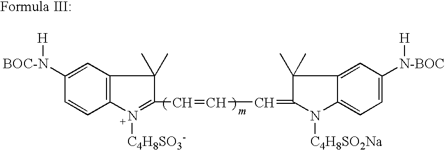

According to the invention, in Formula II, preferably m is 1, 2 or 3. According to the invention, preferably m is 3. In Formula III, BOC stands for t-butoxycarbonyl.

According to the invention, preferably the pigment is a merocyanine pigment. According to the invention, preferably the pigment is MC6.1. According to the invention, the pigment MC6.2 is preferred. According to the invention, the pigment MC6.1-iodoacetamide is preferred. According to the invention, the pigment MC6.2-iodoacetamide is preferred.

According to the invention, preferably the pigment has a structure as per Formula IV:

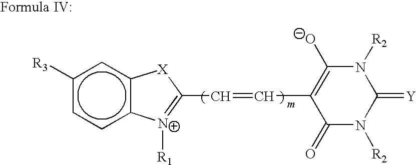

X, Y, R₁, R₂ and R₃ in Formula IV preferably can, in accordance with the invention, be various groups. In accordance with the invention, preferably m is 1, 2 or 3. According to the invention preferably m is 3. According to the invention, preferably X is S, O, an N—R or an isopropylide group. According to the invention preferably Y is S, O, an N—R or an isopropylide group. According to the invention, X is preferably an isopropylide group. According to the invention, Y is preferably sulfur. According to the invention, Y is preferably oxygen. According to the invention, R₁ is preferably a methyl group. According to the invention R₂ is preferably an ethyl group. According to the invention, R₂ is preferably a butyl group. According to the invention, R₃ is preferably IA, whereby IA stands for iodoacetamide.

In accordance with the invention, preferably the pigment has a structure as per Formula V:

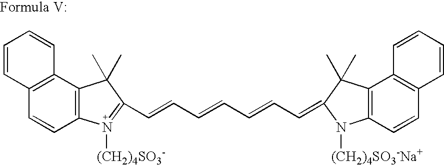

According to the invention, the pigment is preferably a derivative of the pigment as per Formula V.

According to the invention, preferably, the pigment has a structure as per Formula VI:

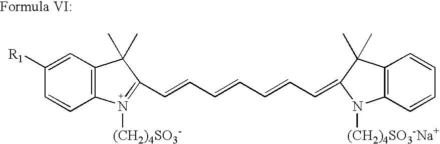

According to the invention, preferably R₁ in Formula VI is isothiocyanate.

According to the invention, the pigment preferably has a structure as per Formula VII:

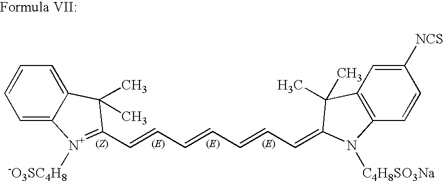

According to the invention, a color-marked oligosaccharide or polysaccharide preferably comprises an agent with Formula VIII.

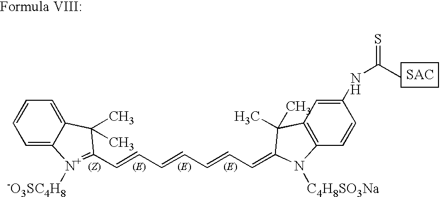

whereby SAC is an oligosaccharide or polysaccharide.

According to the invention, preferably a color-marked oligosaccharide or polysaccharide comprises an agent with Formula IX:

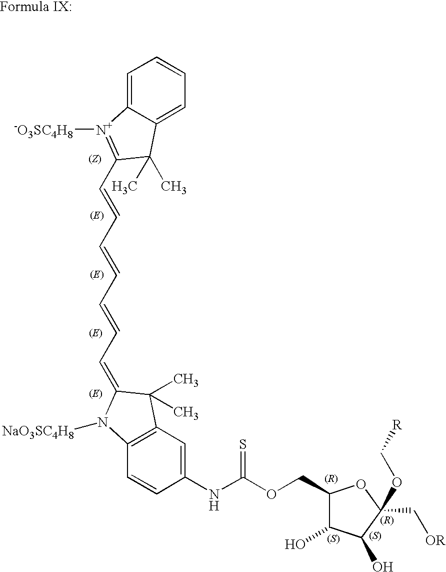

whereby R stands for at least one additional fructose unit.

In accordance with the invention, the oligosaccharide or polysaccharide is preferably coupled directly to the pigment. In accordance with the invention, the oligosaccharide or polysaccharide is preferably coupled to the pigment with isothiocyanate. In accordance with the invention, preferably isothiocyanate is the coupling between the pigment and the oligosaccharide or polysaccharide. In accordance with the invention, preferably the isothiocyanate is a component of the pigment. In accordance with the invention, the isothiocyanate is not a component of the pigment. In accordance with the invention the pigment is coupled to an OH group of a fructose unit or a glucose unit of the oligosaccharide or polysaccharide. Thereby, the pigment can be coupled to any free OH group of a fructose unit or a glucose unit. In accordance with the invention, the pigment is preferably coupled to a terminal fructose unit of the oligosaccharide or polysaccharide. In accordance with the invention, the pigment is preferably coupled to a terminal glucose unit of the oligosaccharide or polysaccharide. In accordance with the invention, the pigment is preferably coupled to a central fructose unit of the oligosaccharide or polysaccharide.

The invention also solves the problem on which it is based by providing a procedure for the synthesis of a color-marked oligosaccharide or polysaccharide, particularly of an oligosaccharide or polysaccharide in accordance with the invention. As per the invention, preferred is a procedure for the synthesis of the color-marked oligosaccharide or polysaccharide according to the invention, whereby a) the oligosaccharide or the polysaccharide is dissolved in a solvent, b) a hydride is added to the dissolved oligosaccharide or polysaccharide, particularly sodium hydride, c) a dissolved pigment, particularly a fluorescent pigment is added, and d) the color-marked oligosaccharide or polysaccharide is isolated as a solid substance.

It is preferred in accordance with the invention that the hydride in step b) is sodium hydride. Preferred in accordance with the invention, the hydride in step b) is sodiumborohydride, i.e. NABH₄. Preferred in accordance with the invention, the hydride in step b) is lithiumaluminiumhydride, i.e. LiAlH₄

The invention also concerns the color-marked oligosaccharides or polysaccharides that are synthesized using this procedure.

In accordance with the invention, the pigment is preferably a fluorescent pigment, preferentially a fluorescent pigment that was named above. In accordance with the invention, preferably the pigment is a previously mentioned pigment.

In accordance with the invention, preferably the solvent in step a) is a polar solvent. In accordance with the invention, preferably the solvent in step a) is an aprotic solvent. In accordance with the invention, preferably the solvent in step a) is a polar aprotic solvent. In accordance with the invention, preferably, the solvent in step a) is an anhydrous dimethylformamide.

In accordance with the invention, preferably, the color-marked oligosaccharide or polysaccharide in step d) is isolated by the addition of watery ammonium chloride, subsequent extraction followed by lypophilization.

In accordance with the invention, the color-marked oligosaccharide or polysaccharide in step d) is purified by recrystallization and/or gel filtration.

The invention also concerns color-marked oligosaccharides or polysaccharides that were synthesized using a conventional procedure.

The invention also concerns the use of a color-marked oligosaccharide or polysaccharide in accordance with the invention for the determination of kidney function. The invention also concerns use of a color-marked oligosaccharide or polysaccharide for the determination of the glomerular filtration rate. The invention also concerns use of a color-marked oligosaccharide or polysaccharide in accordance with the invention for the determination of renal clearance.

The use of the color-marked oligosaccharides or polysaccharides in accordance with the invention for the determination of kidney function and/or as a diagnostic agent provides non-invasive detection. This contributes to the reduction of physical interference with the individual that is to be examined.

The invention also concerns the use of a color-marked oligosaccharide or polysaccharide in accordance with the invention for the synthesis of an agent for the determination of kidney function. The invention also concerns the use of a color-marked oligosaccharide or polysaccharide in accordance with the invention for the synthesis of an agent for the determination of the glomerular filtration rate. The invention also concerns use of a color-marked oligosaccharide or polysaccharide for the synthesis of an agent for the determination of renal clearance.

The invention also concerns diagnostic agents, particularly for the determination of kidney function, containing a color-marked oligosaccharide or polysaccharide in accordance with the invention. Preferable in accordance with the invention is that the diagnostic agent serves the determination of kidney function. In accordance with the invention, preferably the diagnostic agent is for the determination of the glomerular filtration rate. In accordance with the invention, preferably, the diagnostic agent is for the determination of renal clearance.

In accordance with the invention, the diagnostic agent preferably contains a color-marked oligosaccharide or polysaccharide. In accordance with the invention, preferably the diagnostic agent contains at least two different color-marked oligosaccharides or polysaccharides.

According to the invention, preferably the diagnostic agent contains substances selected from the group consisting of additives, carrier substances, excipients and mixtures of such. In accordance with the invention, preferably the diagnostic agent contains additives. In accordance with the invention, preferably the diagnostic agent contains carrier substances. In accordance with the invention, preferably the diagnostic agent contains excipients. In accordance with the invention, preferably the diagnostic agent contains physiologically compatible buffer substances.

The color-marked oligosaccharide or polysaccharide in accordance with the invention is preferably used in accordance with the invention, as a component of a parenterally administered diagnostic agent. For the synthesis of the diagnostic agent it is preferred in accordance with the invention, that the color-marked oligosaccharide or polysaccharide in accordance with the invention is dissolved in water, especially water for injection purposes as per DAB10, or physiological cooking salt solution, especially isotonic sodiumchloride solution. Preferably, in accordance with the invention, the concentration of the diagnostic agent of the color-marked oligosaccharide or polysaccharide according to the invention is in the range of 10 to 250 mg/ml, particularly between 25 and 125 mg/ml.

The invention also concerns a diagnostic procedure, particularly for the determination of kidney function, characterized by, that a color-marked oligosaccharide or polysaccharide in accordance with the invention or a diagnostic agent in accordance with the invention is administered. Preferred in accordance with the invention, the diagnostic process is for the determination of kidney function. Preferred according to the invention, the diagnostic procedure is for the determination of the glomerular filtration rate. Preferred according to the invention, the diagnostic procedure is for the determination of renal clearance.

In accordance with the invention, preferably the color-marked oligosaccharide or polysaccharide in accordance with the invention or the diagnostic agent in accordance with the invention is administered to a vertebrate, particularly preferred a mammal, especially a human being. In accordance with the invention, preferably the diagnostic procedure is used on a mammal. In accordance with the invention, preferably the diagnostic procedure is used on a human being.

In accordance with the invention, preferably, the diagnostic procedure is performed with a color-marked oligosaccharide or polysaccharide according to the invention. Preferably, according to the invention the diagnostic procedure is performed with at least two different color-marked oligosaccharide or polysaccharides according to the invention.

For the evaluation of kidney function, preferably, in accordance with the invention, the chronological sequence of the concentration of the color-marked oligosaccharide or polysaccharide in accordance with the invention is determined in the blood after parenteral administration of a dosage of the color-marked oligosaccharide or polysaccharide.

In accordance with the invention, preferably the diagnostic procedure includes a measurement of fluorescence. Preferred in accordance with the invention, the diagnosis takes place by determining the concentration of the color-marked oligosaccharide or polysaccharide according to the invention by measuring fluorescence by methods from prior art (compare, for example, Sohtell et al., Acta Physiol. Scand. 119 (1983); page 313 to 316, Lorenz and Gruenstein, Am. J. Physiol 276 (1999) page 172-177).

The measurement of fluorescence has the advantage of high sensitivity and quickness of measurement.

In accordance with the invention, preferably, the diagnosis takes place transcutaneously.

In accordance with the invention, preferred is a non-invasive diagnostic process for the determination of kidney function, comprising the irradiation of light onto the skin of the mammal that is to be examined, particularly human beings, and the capturing of the fluorescent light generated by the skin, that contains a color-marked oligosaccharide or polysaccharide according to the invention or by a diagnostic agent that contains a color-marked oligosaccharide or polysaccharide according to the invention.

Preferably, the measurement of fluorescence of the color-marked oligosaccharide or polysaccharide in accordance with the invention is performed in vitro, for example, in blood samples. Preferably, in accordance with the invention, no enzymatic pre-treatment is performed for the measurement of fluorescence.

In accordance with the invention, preferably the measurement of fluorescence is performed with conventional standard devices. Preferred, according to the invention, the measurement of fluorescence of the color-marked oligosaccharide or polysaccharide is preformed with a non-invasive method of detection. Non-invasive detection methods are methods that permit detection of the oligosaccharide or polysaccharide in accordance with the invention in the tissue or in bodily fluids without prior drawing of a sample, for example, drawing a blood sample.

In accordance with the invention, preferably the measurement of the fluorescence of the color-marked oligosaccharide or polysaccharide according to the invention takes place in the tissue or in bodily fluids. In accordance with the invention, preferably a measurement of florescence is used in which light is irradiated onto the skin of the individual that is to be examined to excite fluorescence and the fluorescent light that is generated by the skin is detected. In accordance with the invention, this preferably takes place with a non-invasive detector head. In accordance with the invention, preferably a light source of the detector head, for example, a laser emitting in the UV range, shines on the skin through fiber optics and excites the pigments of the oligosaccharide or polysaccharides color-marked in accordance with the invention that are contained in it to fluoresce. In accordance with the invention, preferably, the fluorescent light is adsorbed by fiber optics and measured with a corresponding detector, for example, a CCD spectrograph. The light source and/or detector can thereby be integrated into the detector head or be located outside the detector head. In accordance with the invention, preferably, the detector head is placed on the skin of the individual to be examined with an adhesive, for example, a transparent adhesive film, and remains there for the entire duration of the measurement.

In accordance with the invention, preferably the non-invasive measurement of the color-marked oligosaccharide or polysaccharide in accordance with the invention is performed continuously over a longer period of time. In accordance with the invention, preferably, the non-invasive measurement is performed over a clinically relevant period of measurement. In accordance with the invention, preferably, the non-invasive measurement is performed over a period of 2 to 4 hours, particularly approximately 3 hours, particularly 3 hours.

In accordance with the invention, the dosage of the color-marked oligosaccharide or polysaccharide according to the invention that is used as diagnostic agent is 5 to 200 mg of substance per kg of body weight, especially preferred 5 to 100 mg of substance per kg of body weight, more preferred 5 to 50 mg of substance per kg of body weight, preferably 5 to 20 mg of substance per kg of body weight.

Additional advantageous embodiments of the invention result from the subordinate claims.

The following examples explain the invention; however, they do not limit it.

### EXAMPLE 1

### Preparation of the Oligosaccharides and Polysaccharides for Color-Marking

Starting materials used:

The starting materials used Beneo™GR and Beneo™95 are made by the company Beneo-Orafti (Tienen, Belgium). These are products that are used in foods that are made of inulin, which is made from chicory (chicorium intybus).

Beneo™GR: Beneo™GR consists of approximately 92% inulin (oligofructose, DP distribution see Table 1) and approximately 8% of glucose (G), fructose (F) and sucrose (GF).

Beneo™95: Beneo™95 consists of approximately 95% inulin (oligofructose, DP distribution see Table 1) and approximately 5% of glucose (G), fructose (F) and sucrose (GF). The table clearly shows the larger share of shorter fructose chains compared to Beneo™GR.

TABLE 1
Composition of the inulins that are used as starting materials

| DP | Beneo ™ 95 | Beneo ™ GR |
|---|---|---|
| G, F, GF | 5 | 8 |
| 2-5 | 77 | 8 |
| 6-10 | 18 | 17 |
| 2-9 | 95 | 25 |
| >10 | 0 | 67 |

### Preparative Gel Chromatography

For separating the molecules of the desired chain length, Size Exclusion Chromatography (SEC) is used, that is also called Gel-Permeations-Chromatography (GPC).

The detection of the substances separated in the column preferably takes place with a Refraction Index Detector (RI) that measures the deviation/change in direction that is experienced by a ray of light when it enters an optically different medium at an angle. The RI detector emits a voltage in mV, that is entered against time (chromatogram).

As separation material, “Toyoperl HW-40 S” (Tosoh Bioscience, D-70567 Stuttgart, Germany, molecular structure: hydroxylated methacrylate polymer, average particle size: 30 μm, particle size distribution: 20-40 μm, exclusion limit: up to 10,000 Dalton, average pore size: 50 Å (0.005 μm) height of separation columns: 125 cm, diameter of separation columns: 10 cm).

### Separation Conditions:

| | |
|---|---|
| Volume gel bed: | 3 × 8 L = 24 L |
| Temperature: | 60° C. |
| Eluent: | fully desalinated, degassed water |
| Flow rate: | 12 ml/min |
| Objective: | respectively 150 ml at 10% (w/w) TS |

Analysis with HPAEC:

The analysis of all probes takes place with HPAEC (High Pressure Anion Exchange Chromatography). The stationary phase is a strongly basic anion exchanger resin, to which various anions bind at various strength. As the hydrocarbons that are to be analyzed here are weak acids, these are present in the basic range as anions and can be separated and detected dependent on their pKs value by gradient elution. The reaction time increases from the monosaccharides to the polysaccharides.

The detection takes place by pulsed amperometry (PAD) that works at three different voltages. The first voltage causes an oxidation of the ions in the probe at a gold electrode, whereby a voltage is generated that is equivalent to the concentration of the respective probe ions. As a result of the effect of the second voltage, the electrode surface is oxidized. Upon the effect of the third voltage, the electrode surface is reduced to gold, which simultaneously cleans the electrode.

TABLE 2
Parameter of the HPAEC Analysis

| Parameter | Condition/Device |
|---|---|
| Column | Pre-column: Carbo-Pac ™, PA 1 Guard |
| | 4 × 50 mm |
| | Separation column: Carbo-Pac ™, PA 1 |
| | 4 × 250 mm |
| Eluent | 1 M NaOH, 1 M sodium acetate, |
| | fully desalinated water |
| Gradient | within 60 minutes: |
| | [2-45% sodium acetate + |
| | 88-45% fully desalinated water] + |
| | 10% NaOH |
| Pressure | 70-100 bar |
| Flow | 1 ml/min |
| Injection volume | 20 μL |
| Probe concentration | 10-50 ppm in buffer |
| Buffer | .1% toluol in fully desalinated water |
| Elution pump | Dionex gradient pump |
| Probe objective | Dionex auto sampler |
| Detection | Pulsed amperometric detection (PAD) |
| Integration | HP ChemStation |
| Sensitivity | 1 μA |

Chromatographic Separation of a Fructo-Oligosaccharide Mixture in the Range of DP 5 to DP 10 (“DP5+”) from Inulin (Beneo™95):

The goal is the synthesis of an oligofructane mixture that contains primarily only fructanes with a molecular size between DP 5 and DP 10. As Beneo™95 contains up to 95% fructanes in the range of DP 2 and DP 9 (see Table 1), only the low molecular range <DP 5 must be separated chromatographically.

For 11.7 g TS product, 6 separations were performed in a separation system (conditions: see above) in which respectively 150 ml at 10% (w/w) TS were put in the column. In total, 90 g TS Beneo™95 were dissolved and chromatographed.

After separation, the product solution was captured in 7 different fractions. As it is known that the larger molecules leave the column first, the fractions were cut narrower in this range than later in the range in which the smaller molecules were eluted. The composition of the fractions was analyzed by HPAEC. It is only possible to associate the peaks with individual chain lengths by HPAEC analysis. The fructose tetramer F₄ that already is no longer a part of the target compound appears in fraction 4 for the first time. In fraction 7, the share of the smaller molecules was already so large that it could not be used anymore and was discarded. Fractions 2 and 6 were combined and concentrated in the rotary evaporator at 40° C. and 40 mbar to 10% (w/w) TS.

Bleaching with charcoal and lyophilization.

The solution that was obtained by preparative gel chromatography and concentrated shows a light yellow coloring, which can be removed with charcoal. To do so, the solution is poured at room temperature into a glass column with a frit bottom that is filled with 250 ml charcoal (Epibon MC-h 12-40, Donau Carbon GmbH & Co. KG, Frankfurt), and the bleached solution is captured. As pre-treatment, the charcoal was washed intensively with hot, fully desalinated water. As the charcoal, in addition to the colored substances also adsorbs fructanes, the yield of bleaching is 48% (relative to the dry substance used).

Because as a result of the bleaching, the solution is again diluted, it must be concentrated in preparation of lyophilization in the rotary evaporator at 40° C. and 40 mbar to 10% (w/w) TS. The concentrated solution is frozen at −20° C. and subsequently freeze-dried at 0.6 mbar. A total of 11.7 g of freeze-dried product was obtained in the experiment.

In the HPAEC chromatogram of the synthesized product it can be clearly seen that compared to the starting material Beneo™95, the smaller molecules (here <DP5) with a retention time <18 are no longer present.

### Composition:

TABLE 3
Composition of the product DP 5 to DP 10
(“DP5+”).

| Fructo-Oligosaccharide | % | |
|---|---|---|
| Inulotriose (F-3) | 0 | Residual |
| Nystose (GF3) | .6 | |
| Inulotetraose (F-4) | 1.8 | |
| F-Nystose (GF-4) | 16.3 | |
| Inulopentaose (F-5) | 30.1 | 89.6% |
| F-F Nystose (GF-%) | 13.9 | Target Products |
| Inulohexaose (F-6) | 21.6 | |
| F-F-F-Nystose (GF-6) | 7.7 | |
| Rest | 8.1 | Residual |
| Total | 100 | |

Chromatographic Separation of a Fructo-Oligosaccharide Fraction with DP 10 (“DP 10”) from Inulin (Beneo™GR):

The goal is the synthesis of a polyfructane fraction that contains primarily molecules at a size of DP 10. As starting material, Beneo™GR was used (see Table 1)

For 4.7 g of TS product, 11 separations were performed in a separation system (conditions: see above), in which respectively 150 ml at 10% (w/w) TS Beneo™GR were put in the column. In total, 225 g of TS Beneo™GR were dissolved and chromatographed. The yield is 2%. The purity related to DP 10 is approximately 30%.

Chromatographic Separation of a Fructo-Oligosaccharide Mixture in the Range of >DP 10 (“DP10+”) from Inulin (Beneo™GR):

The goal is the synthesis of an oligofructane mixture that contains primarily only fructanes with a molecular size of between DP 10 and DP 15. As Beneo™GR is composed of fructanes in the range of DP 2 to approximately DP 60, (Table 1 or manufacturer specifications from the firm Beneo-Orafti (Tienen, Belgium)), prior to the chromatographic separation with isopropanol, the DP range starting at approximately 15 DP was precipitated and filtered out.

Isopropanol precipitation:
110 g Beneo™GR are weighed into a glass beaker in 1,100 g of fully desalinated water
for the precipitation of the higher molecular components, 2,200 g of isopropanol are added and it is stirred for 10 minutes
the milky solution is first sucked off with a “Schwarzbandfilter” then with a 0.45 μm filter
in the rotary evaporator, the filtrate is concentrated at 50° C. to a third of the starting quantity (370 g) and can then be used for chromatography
obtained were 55.6 g TS oligofructane mixture

For 3.3 g of TS product DP 10+, 2 separations (conditions see above) were performed in a separation system in which respectively

150 ml at 10% (w/w) TS of the oligofructane mixture was put in the column. The yield is 3%.

TABLE 4
Composition of the product DP 10 to DP 15 (“DP10+”).

| Fructo-Oligosaccharide | % | |
|---|---|---|
| Nystose (GF-4) | 1.6 | Residual |
| Inulopentaose (F-5) | 2.9 | |
| DP-10 (GF9) | 14.8 | 82.7% |
| DP-11 (GF10) | 29.0 | Target Products |
| DP-12 (GF11) | 20.5 | |
| DP-13 (GF12) | 12.2 | |
| DP-14 (GF13) | 6.2 | |
| Rest | 12.8 | Residual |
| Total | 100 | |

### EXAMPLE 2

### Synthesis and Purification if ICG-ITC-Marked Sinistrin

2 g sinistrin is dissolved in 80 ml DMF at 35° C. under N₂ gas in a three-neck flask under reflux cooling
let solution cool to room temperature
0.44 g NaH is added in small parts (solution becomes slightly yellow and viscous)
solution is stirred at 45° C. for 30 minutes
solution is stirred at 55° C. for 30 minutes
0.072 g ICG-ITC dissolved in 35 ml DMF is added at once
solution is stirred at 55° C. for 60 minutes
stir over night at room temperature
cool solution to 5° C. and add, while vigorously stirring 0.95 g NH₄CL dissolved in 5 ml H₂O (solution discolors from brown to dark green)
separate solution in rotary evaporator
a green solid substance remains
dissolve product in methanol/H₂O 1/1
precipitate in acetone
re-suspend solid substance in 40 ml methanol/H₂O 1/1
add 2 g silica 60
separate solvent in rotary evaporator
product is put in a column filled with silica60/methanol/toluol (2/1)
impurities are separated with methanol/toluol (2/1)
product is eluted in methanol/H₂O
mix again with silica 60 and elute with the same solvents
product is frozen at −80° C.

in lyophilization, a green solid substance is created.

1-27. (canceled)

28. A color-marked oligosaccharide or polysaccharide, comprising an oligosaccharide or polysaccharide coupled with a pigment, whereby the pigment has an emission maximum of at least 725 nm and at most 875 nm.

29. A color-marked oligosaccharide or polysaccharide, comprising an oligosaccharide or polysaccharide coupled with a cyanine pigment.

30. The color-marked oligosaccharide or polysaccharide of claim 29, wherein the cyanine pigment has an emission maximum of at least 725 nm and at most 857 nm.

31. The color-marked oligosaccharide or polysaccharide of claim 28, wherein the pigment is a fluorescent pigment.

32. The color-marked oligosaccharide or polysaccharide of claim 28, wherein the pigment is a mono-functional or a bi-functional isothiocyanate cyanin pigment.

33. The color-marked oligosaccharide or polysaccharide of claim 28, wherein the pigment is an indocyanine green derivative.

34. The color-marked oligosaccharide or polysaccharide of claim 28, with the structure
CY-ITC-SAC,
whereby CY is a cyanine pigment; ITC is isothiocyanate; and SAC is an oligosaccharide or a polysaccharide.

35. The color-marked oligosaccharide or polysaccharide according to claim 28, wherein an indocyanine green derivative is synthesized from indocyanine green and isothiocyanate.

36. The color-marked oligosaccharide or polysaccharide according to claim 28, comprising an agent with the formula VIII, 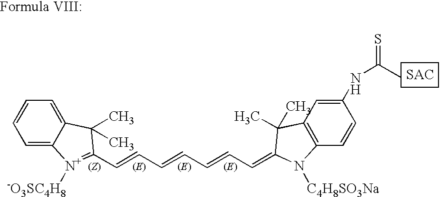
whereby SAC is an oligosaccharide or polysaccharide.

37. The color-marked oligosaccharide or polysaccharide of claim 28, wherein the oligosaccharide or the polysaccharide has a DP of 2 to 65.

38. The color-marked oligosaccharide or polysaccharide of claim 28, wherein the oligosaccharide or the polysaccharide is branched.

39. The color-marked oligosaccharide or polysaccharide of claim 28, wherein the oligosaccharide or the polysaccharide is not branched.

40. The color-marked oligosaccharide or polysaccharide of claim 28, wherein the oligosaccharide or the polysaccharide is selected from a group comprising: oligofructanes, polyfructanes, inulin, sinistrin and insoluble maltodextrines or dextrines that have, in addition to the α-1,4 bond at least one α-1,3, α-1,2 and/or an α-1,6 bond.

41. The color-marked oligosaccharide or polysaccharide of claim 28, wherein the oligosaccharide or the polysaccharide is sinistrin.

42. The color-marked oligosaccharide or polysaccharide of claim 28, wherein the oligosaccharide or the polysaccharide is an oligofructane.

43. The color-marked oligosaccharide or polysaccharide of claim 28, wherein the oligosaccharide or the polysaccharide is a polyfructane.

44. The color-marked oligosaccharide or polysaccharide of claim 28, wherein the oligosaccharide is 1-F-fructofuranosyl-d-nystose.

45. The color-marked oligosaccharide or polysaccharide of claim 28, wherein the oligosaccharide or the polysaccharide is insoluble maltodextrin that has, in addition to the α-1,4 bond, at least one α-1,3, α-1,2 and/or an α-1,6 bond.

46. The color-marked oligosaccharide or polysaccharide of claim 28, wherein the oligosaccharide or the polysaccharide is an insoluble dextrin that has, in addition to the α-1,4 bond, at least one α-1,3, α-1,2 and/or an α-1,6 bond.

47. A procedure for the synthesis of a color-marked oligosaccharide or polysaccharide according to claim 28, wherein
a) the oligosaccharide or the polysaccharide is dissolved in a solvent;
b) sodium hydride is added to the dissolved oligosaccharide or polysaccharide;
c) a dissolved pigment, particularly a fluorescent pigment is added; and
d) the color-marked oligosaccharide or polysaccharide is isolated as a solid substance.

48. The procedure according to claim 47, wherein the solvent in step a) is anhydrous dimethylformamide.

49. The procedure according to claim 47, wherein the color-marked oligosaccharide or polysaccharide in step d) is isolated by adding watery ammonium chloride, subsequent extraction followed by lyophilization.

50. Use of a color-marked oligosaccharide or polysaccharide according to claim 28 for the determination of kidney function.

51. Use of a color-marked oligosaccharide or polysaccharide according to claim 28 for the synthesis of an agent for kidney function determination.

52. A diagnostic agent for determining kidney function, containing a color-marked oligosaccharide or polysaccharide according to claim 28 and substances selected from a group comprising additives, carrier substances, excipients and mixtures thereof.

53. A diagnostic procedure for the determination of kidney function comprising administering to a vertebrate a color-marked oligosaccharide or polysaccharide according to claim 28.

54. A diagnostic procedure for the determination of kidney function comprising administering to a vertebrate a diagnostic agent according to claim 52.
